(19) United States
(12) Patent Application Publication
(10) Pub. No.: US 2023/0285350 A1
(43) Pub. Date: Sep. 14, 2023

(54) FORMULATIONS FOR TETRACAINE AND LIDOCAINE

(71) Applicant: LYOTIP, INC., Camarillo, CA (US)

(72) Inventor: Byeong Seon Chang, Thousand Oaks, CA (US)

(73) Assignee: LYOTIP, INC., Camarillo, CA (US)

(21) Appl. No.: 18/016,656

(22) PCT Filed: Jul. 15, 2021
(86) PCT No.: PCT/US2021/041893

§ 371 Date: Jan. 17, 2023

Related U.S. Application Data
(60) Provisional application No. 63/052,301, filed on Jul. 15, 2020.

(51) Int. Cl. A61K 31/245; A61K 9/08; A61K 31/167; A61K 47/26; A61K 31/137

(52) U.S. Cl. CPC A61K 31/245; A61K 9/08; A61K 31/167; A61K 47/26; A61K 31/137

(57) ABSTRACT

Tetracaine is a common anesthetic. Because of its limited stability in solution, it is generally lyophilized for storage and shipping. Likewise, any combination products containing tetracaine need to be lyophilized. Embodiments include a stable formulation for tetracaine or its combination with other anesthetics like lidocaine that obviates the need for lyophilization. Also included are methods of stabilizing an anesthetic in solution by adding a non-ionic tonicity modifier and adjusting the pH.

### RELATED APPLICATIONS

This application claims priority and is entitled to the filing date of U.S. provisional patent application Ser. No. 63/052,301 filed on Jul. 15, 2020. The contents of the aforementioned application are incorporated herein by reference.

### FIELD OF INVENTION

This invention relates generally to formulations for anesthetics, and more specifically, to formulations that provide stability to tetracaine and lidocaine in solution.

### BACKGROUND

Anesthesia can be defined as insensitivity to pain, particularly as artificially induced by the administration of gases or the injection of drugs before a surgical operation. Anesthesia enables medical procedures that would otherwise cause severe or intolerable pain or would otherwise be technically unfeasible. There are three general categories of anesthesia. General anesthesia suppresses central nervous system activity and results in unconsciousness and total lack of sensation; Sedation suppresses the central nervous system to a lesser degree, inhibiting both anxiety and creation of long-term memories without resulting in unconsciousness; Local anesthesia blocks transmission of nerve impulses from a specific part of the body.

Local anesthetic agents prevent transmission of nerve impulses without causing unconsciousness. They act by reversibly binding to fast sodium channels from within nerve fibers, thereby preventing sodium from entering the fibers, stabilizing the cell membrane and preventing action potential propagation. Each of the local anesthetics have the suffix “-caine” in their names. Local anesthetics can be either ester- or amide-based. Amides are generally used within regional and epidural or spinal techniques, due to their longer duration of action, which provides adequate analgesia for surgery, labor, and symptomatic relief. Ester-type topical anesthetics are metabolized by plasma cholinesterase and other nonspecific esterases, while amide anesthetics are primarily metabolized in the liver via microsomal enzymes.

Tetracaine, also known as amethocaine, is an ester local anesthetic often used to numb the eyes, nose, or throat. It can also be applied to the skin before starting an intravenous to decrease pain from the procedure. It is typically applied as a liquid to the area. It works by blocking the sending of nerve impulses. Onset of effects when used in the eyes is within 30 seconds and last for less than 15 minutes.

Lidocaine, also known as lignocaine, is an amide local anesthetic that is often used to numb tissue in a specific area. It can also used to treat ventricular tachycardia and to perform nerve blocks. Lidocaine mixed with a small amount of adrenaline (epinephrine) is available to allow larger doses for numbing, to decrease bleeding, and to make the numbing effect last longer. When used as an injectable, lidocaine typically begins working within four minutes and lasts for half an hour to three hours. Lidocaine mixtures may also be applied directly to the skin or mucous membranes to numb the area.

Epinephrine, also known as adrenaline, is often used in combination with local anesthetics (Las). Epinephrine can decrease the rate of systemic absorption, prolong the duration of action and reduce the risk of systemic toxicity. When used with infiltration anesthesia it can reduce bleeding in the operative field. When used with tetracaine, it can prolong the numbing effect and motor block effect of the anesthetic by up to an hour.

The efficacy and potency of an anesthetic depend in part on its stability during shipping and storage. Tetracaine in solution is be prone to degradation, particularly when agitated or exposed to heat. Transportation and storage at ambient or lower temperatures can also lead to degradation. For this reason, storage and shipping in solution is impractical. Therefore, tetracaine is typically stored and shipped as a lyophilized product.

Lyophilization involves drying a solution at low temperature. It can improve the stability of the products that have limited stability in solution. However, there are shortcomings with lyophilization. Handling and processing times increase. The equipment necessary to lyophilize industrial can be expensive and costly to operate and maintain. Further, a healthcare professional must properly reconstitute the produce in sterile diluent for proper use.

Combining anesthetic agents with epinephrine has become a common practice because of the synergistic effect of combined agents and potentially lower side effects. For example, LET solution is often applied to lacerated skin before suturing. LET is commercially available as a kit with four solid ingredients: lidocaine hydrochloride, epinephrine bitartrate, tetracaine hydrochloride and sodium metabisulfite. The ingredients are lyophilized because they degrade when suspended in solution. A health care professional must resuspend the ingredients in sterile solution prior to use. This must be done properly to ensure the solution has the intended concentration and activity. Thereafter, any remaining LET is solution is discarded if not used within a short time thereafter.

The combination product of lidocaine and epinephrine is available as a liquid product. However, a product with tetracaine is not yet available as a liquid product due to its instability in solution. Accordingly, there is a need for a formulation that improves the stability of tetracaine as well as other anesthetics like lidocaine and related compounds in solution. A formulation with stability-conferring properties could lower costs, allow convenient administration, and increase accessibility to anesthetics, particularly in rural and under-served areas.

There is, therefore, a need for methods to make improved formulations for keeping both tetracaine and lidocaine stable in solution. One aspect of the invention is a formulation that provides a stable solution for storage and transportation of an anesthetic (i.e. tetracaine) without lyophilization. Another aspect is a method of stabilizing a solution with tetracaine to prevent or reduce degradation.

### SUMMARY

The inventions described and claimed herein have many attributes and embodiments including, but not limited to, those set forth or described or referenced in this brief summary. The inventions described and claimed herein are not limited to, or by, the features or embodiments identified in this Summary, which is included for purposes of illustration only and not restriction.

Aspects of the invention also include a formulation for stabilizing an anesthetic or combination of anesthetic agents. The anesthetic can be an ester or an amide anesthetic. The anesthetic can be one or more of procaine, amethocaine, cocaine, benzocaine, tetracaine, lidocaine, prilocaine, bupivicaine, levobupivacaine, ropivacaine, mepivacaine, dibucaine, and etidocaine.

Additional aspects of the invention include a stabilizing formulation for an anesthetic, wherein the stability of the anesthetic is determined by (a) HPLC, (b) mass spectrometry, (c) UPLC with HRMS or (c) an activity assay.

The experimental examples described herein demonstrate that tetracaine is more stable in solution when formulated with non-ionic tonicity modifiers. Further, it is more stable at low pH (about 3.5-4.5) with a non-ionic tonicity modifier such as sorbitol. Accordingly, embodiments include a formulation for stabilizing tetracaine that has a pH of about 3.5 and includes a non-ionic tonicity modifier such as sorbitol.

The experimental examples described herein also demonstrate that lidocaine is not stable at low pH (about 3.5-4.5) in normal saline but becomes stable when the tonicity modifier is replaced with a non-ionic tonicity modifier. Accordingly, embodiments include a formulation for stabilizing lidocaine that has a pH of about 3.5 and includes a non-ionic tonicity modifier such as sorbitol.

The experimental examples described herein also demonstrate both tetracaine and lidocaine can be formulated together at pH 3.5 with a non-ionic tonicity modifier such as sorbitol.

Another embodiment is a stable topical anesthetic formulation that includes (a) tetracaine, (b) lidocaine and (c) sorbitol in 10 mM sodium acetate buffer with a pH of about 3.5-4.5. The tetracaine can be about 0.5% w/w and the lidocaine can be about 4% w/w. The sorbitol is at about 5% w/w.

Also described herein are methods of improving the stability of an anesthetic solution that includes steps of (a) providing an anesthetic, (b) adding a non-ionic tonicity modifier and (c) adjusting the pH of the anesthetic solution to about 3.5-4.5. The anesthetic can be tetracaine or lidocaine and the tonicity modifier can be one or more carbohydrate compounds. In an embodiment, the tonicity modifier is non-ionic. In another embodiment, the tonicity modifier is sorbitol.

Another embodiment is a stable topical anesthetic formulation that includes (a) about 4 mg/ml lidocaine, (b) a non-ionic tonicity modifier at isotonic concentration, (c) about 10 mM sodium acetate buffer and (d) water at a pH of about 4.5. Yet another embodiment is a stable topical anesthetic formulation that includes (a) about 0.5 to 2 mg/ml tetracaine, (b) a non-ionic tonicity modifier at isotonic concentration, (c) a buffer and (d) water at a pH of about 3.5-4.5.

Another embodiment is a method of stabilizing a solution of LET, the method comprising steps of (a) adding a non-ionic tonicity modifier, and (b) adjusting the pH of the anesthetic solution to about 3.5-4.5. In one aspect, the non-ionic tonicity modifier is sorbitol. The non-ionic tonicity modifier can be added to the formulation until it reaches about 5% (w/w).

Another embodiment is a stable topical anesthetic formulation that includes (a) an anesthetic, (b) epinephrine and (c) a non-ionic tonicity modifier in 10 mM sodium acetate buffer at a pH of about 3.5-4.5. The non-ionic tonicity modifier can be sorbitol at about 5% w/w. The anesthetic can be an ester, an amide and/or one or more of procaine, amethocaine, cocaine, benzocaine, tetracaine, lidocaine, prilocaine, bupivicaine, levobupivacaine, ropivacaine, mepivacaine, dibucaine and etidocaine. In one aspect, the formulation also includes a permeation enhancer.

Another embodiment is a formulation that includes tetracaine, lidocaine, epinephrine and a non-ionic tonicity modifier in 10 mM sodium acetate buffer at pH about 3.5-4.5. The formulation can confer stability on each of the tetracaine and lidocaine, such that at least 90% of each of the tetracaine and lidocaine is present after 24 months when stored at 25° C. In one aspect, at least 80% of each of the tetracaine and lidocaine is present after 24 months when stored at 25° C. In one aspect, at least 70% of each of the tetracaine and lidocaine is present after 24 months when stored at 25° C.

Aspects of the invention also include a method of treating a human or animal by alleviating pain or numbing nerves. The method can include the administration of an anesthetic for treating the condition wherein the anesthetic is stored in a stabilizing formulation.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 is a graph of the stability of tetracaine over six months when stored at 50° C. in one of four formulations: A3.5N, A3.5S, A4.5N or A4.5S.

FIG. 2 is a graph of the stability of tetracaine over 24 months when stored at 40° C. in each formulation (i.e., A3.5N, A3.5S, A4.5N or A4.5S).

FIG. 3 is a graph of the stability of tetracaine over 24 months when stored at 25° C. in each formulation (i.e., A3.5N, A3.5S, A4.5N or A4.5S).

FIG. 4 is a graph of the stability of tetracaine over 24 months when stored at 5° C. in each formulation (i.e., A3.5N, A3.5S, A4.5N or A4.5S).

FIG. 5 is a graph of the stability of lidocaine over 24 weeks when stored at 50° C. in each formulation (i.e., A3.5N, A3.5S, A4.5N or A4.5S).

FIG. 6 is a graph of the stability of lidocaine over 24 months when stored at 40° C. in each formulation (i.e., A3.5N, A3.5S, A4.5N or A4.5S).

FIG. 7 is a graph of the stability of lidocaine over 24 months when stored at 25° C. in each formulation (i.e., A3.5N, A3.5S, A4.5N or A4.5S).

FIG. 8 is a graph of the stability of lidocaine over 24 months when stored at 5° C. in each formulation (i.e., A3.5N, A3.5S, A4.5N or A4.5S).

FIG. 9A is a graph of the stability of tetracaine at one week, three weeks, four weeks, nine weeks, six months and twenty-four months when stored in A3.5S formulation at various temperatures (i.e., 5° C., 25° C., 40° C. and 50° C.).

FIG. 9B is a graph of the stability of lidocaine at one week, three weeks, four weeks, nine weeks, six months and twenty-four months when stored at in A3.5S formulation at various temperatures (i.e., 5° C., 25° C., 40° C. and 50° C.).

FIG. 10A is a graph of the stability of tetracaine at one week, three weeks, four weeks, nine weeks, six months and twenty-four months when stored at in A4.5S formulation at various temperatures (i.e., 5° C., 25° C., 40° C. and 50° C.).

FIG. 10B is a graph of the stability of lidocaine at one week, three weeks, four weeks, nine weeks, six months and twenty-four months when stored at in A4.5S formulation at various temperatures (i.e., 5° C., 25° C., 40° C. and 50° C.).

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human. In an embodiment, a “subject” of diagnosis or treatment is a prokaryotic or a eukaryotic cell, a tissue culture, a tissue or an animal, e.g. a mammal, including a human.

The term “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

“An effective amount” refers to the amount of the defined component sufficient to achieve the desired chemical composition or the desired biological and/or therapeutic result. In an embodiment, that result can be the desired pH or chemical or biological characteristic, e.g., stability of the formulation.

The term “lyophilization” refers to a freeze-drying process that removes water from a product after it is frozen and placed under a vacuum. Some of the typical pharmaceutical products that would undergo lyophilization include bulk pharmaceutical/biopharmaceutical ingredient (chemical or biologics found in nature), protein, collagen, peptide, oligonucleotide, chemical API, enzymes, and mAbs.

In an embodiment, as used herein, the terms “treating,” “treatment” and the like are used herein, without limitation, to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

In an embodiment, a “pharmaceutical composition” is intended to include, without limitation, the combination of an active agent with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

As used herein, the term “comprising” is intended to mean that the compositions and methods include the listed elements, but do not exclude other unlisted elements. “Consisting essentially of” when used to define compositions and methods, excludes other elements that alters the basic nature of the composition and/or method, but does not exclude other unlisted elements. Thus, a composition consisting essentially of the elements as defined herein would not exclude trace amounts of elements, such as contaminants from any isolation and purification methods or pharmaceutically acceptable carriers, such as phosphate buffered saline, preservatives, and the like, but would exclude additional unspecified amino acids. “Consisting of” excludes more than trace elements of other ingredients and substantial method steps for administering the compositions described herein. Embodiments defined by each of these transition terms are within the scope of this disclosure and the inventions embodied therein.

The term “HPLC” or “high-performance liquid chromatography” refers to a technique used to separate, identify, and quantify each component in a mixture. A pump passes a pressurized liquid solvent containing the sample mixture through a column filled with a solid adsorbent material. Each component in the sample interacts slightly differently with the adsorbent material, causing different flow rates for the different components and leading to the separation of the components as they flow out of the column. Reversed phase HPLC (RP-HPLC) has a non-polar stationary phase and an aqueous, moderately polar mobile phase. One common stationary phase is a silica which has been surface-modified with RMe2SiCl, where R is a straight chain alkyl group such as C₁₈H₃₇ or C₈₁H₁₇. With such stationary phases, retention time is longer for molecules which are less polar, while polar molecules elute more readily.

The term “tonicity” refers to a measure of the effective osmotic pressure gradient; the water potential of two solutions separated by a semipermeable cell membrane. Tonicity is the relative concentration of solutes dissolved in solution which determine the direction and extent of diffusion. It is commonly used when describing the response of cells immersed in an external solution. Unlike osmotic pressure, tonicity is influenced only by solutes that cannot cross the membrane, as only these exert an effective osmotic pressure. Solutes able to freely cross the membrane do not affect tonicity because they will always equilibrate with equal concentrations on both sides of the membrane without net solvent movement. There are three classifications of tonicity that one solution can have relative to another: hypertonic, hypotonic, and isotonic.

The term “LET” or “Lido-Epineph-Tetra” refers to a topical anesthetic that contains lidocaine, epinephrine, and tetracaine. Commercially available LET solution typically contains lidocaine HCl USP (4%), epinephrine bitartrate USP (180 mg-55% epinephrine and 45% bitartrate), and tetracaine HCl USP (500 mg), with sodium metabisulfite (75 mg) in sterile water for irrigation USP to 100 ml. The stability can be up to six months when refrigerated and up to four weeks when stored at room temperature.

The term “TAC” refers to a mixture of 5% to 12% tetracaine, 0.05% adrenaline, and 4% or 10% cocaine hydrochloride. It can be used in ear, nose, and throat surgery and in the emergency department where numbing of the surface is needed rapidly, especially when children have been injured in the eye, ear, or other sensitive locations. TAC has largely been replaced with LET solution which does not contain cocaine hydrochloride.

The term “permeation enhancer” or “penetration enhancer” refers to substances that promote the absorption of drug through the skin temporarily by transiently enhancing the skin permeability. They can be employed to transfer the delivery of drugs which are ionizable (e.g. timolol maleate) and impermeable (e.g. heparin); to maintain drug levels in blood, to provide higher dose of less potentially active drugs (e.g. oxymorphane), to deliver high molecular weight hormones and peptides and to reduce the lag time of transdermal drug delivery system

Many known and useful compounds and the like can be found in Remington's Pharmaceutical Sciences (13^{th} Ed), Mack Publishing Company, Easton, Pa.—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

As the patients and subjects of the invention method are, in addition to humans, veterinary subjects, formulations suitable for these subjects are also appropriate. Such subjects include livestock and pets as well as sports animals such as horses, greyhounds, and the like.

### DETAILED DESCRIPTION

Local anesthetic agents are often used to reduce pain by preventing transmission of nerve impulses. They act by reversibly binding to fast sodium channels from within nerve fibers, thereby preventing sodium from entering the fibers, stabilizing the cell membrane and preventing action potential propagation.

Local anesthetics can be either ester- or amide-based. Ester local anesthetics include procaine, amethocaine, cocaine, benzocaine and tetracaine. Amide local anesthetics include lidocaine, prilocaine, bupivicaine, levobupivacaine, ropivacaine, mepivacaine, dibucaine and etidocaine. Because of their instability in solution, some like tetracaine are typically lyophilized for storage and shipping.

Tetracaine is a local anesthetic often used to numb the eyes, nose, or throat. It can also be applied to the skin before starting an intravenous to decrease pain from the procedure. Lidocaine is another local anesthetic that is often used to reduce pain or discomfort caused by skin irritations such as sunburn, insect bites, minor cuts, scratches, or burns. LET is a solution that contains lidocaine, epinephrine, and tetracaine.

Because some anesthetic molecules are not stable in liquid or frozen form, they are typically lyophilized. Lyophilization enables longer shelf life and provides a product that is easier to ship. However, lyophilization presents disadvantages. Equipment needed for lyophilization is generally costly and complex. Because of this, the handling and processing time increases. Further, a sterile diluent is needed for reconstitution. A health care professional must properly resuspend the product and ensure it has the intended concentration and activity before administration.

Embodiments of the invention include a formulation for stabilizing an anesthetic or combination of anesthetics. The anesthetic can be an ester or an amide anesthetic, such as tetracaine or lidocaine. Additional embodiments include methods of stabilizing an anesthetic in a solution. The methods described herein can also be used to improve the stability of a medicament in a solution.

### Formulations

The inventor has discovered that adding a non-ionic tonicity modifier and adjusting the pH can improve the stability of tetracaine and lidocaine in solution. Accordingly, aspects of the invention include a stabilizing formulation for a product such as an anesthetic, wherein the formulation is acidic and includes a non-ionic tonicity modifier. In one embodiment, the non-ionic tonicity modifier is dextrose, fructose, galactose, glycerin, sorbitol, mannitol, trehalose, or lactose. The formulation can be stored and shipped as a solution.

In particular, the inventor has discovered that tetracaine is more stable in solution when formulated with non-ionic tonicity modifiers. Further, it is more stable at low pH (around 3.5) with a non-ionic tonicity modifier. Lidocaine is not stable at low pH (around 3.5) in normal saline but becomes stable when the tonicity modifier is replaced with a non-ionic tonicity modifier. Both tetracaine and lidocaine can be formulated together at pH 3.5 with non-ionic tonicity modifier such as sorbitol.

In one embodiment, the candidate formulation (A3.5N) is a solution that includes the following components: lidocaine, tetracaine and sodium chloride in sodium acetate buffer. The pH of the reconstituted solution is approximately 3.5.

More specifically, A3.5N formulation includes the components of Table 1:

TABLE 1
A3.5N (pH 3.5)

| | | |
|---|---|---|
| Sodium acetate buffer | 10 | mM |
| NaCl | 150 | mM |
| Lidocaine | 4 | mg/ml |
| Tetracaine | 2 | mg/ml |

In one embodiment, the candidate formulation (A3.5S) is a solution that includes the following components: lidocaine, tetracaine and sorbitol in sodium acetate buffer. The pH of the reconstituted solution is approximately 3.5.

More specifically, A3.5S formulation includes the components of Table 2:

TABLE 2
A3.5S (pH 3.5)

| Sodium acetate buffer | 10 | mM |
|---|---|---|
| Sorbitol | 5% | |
| Lidocaine | 4 | mg/ml |
| Tetracaine | 2 | mg/ml |

In one embodiment, the candidate formulation (A4.5N) is a solution that includes the following components: lidocaine, tetracaine and sodium chloride in sodium acetate buffer. The pH of the reconstituted solution is approximately 3.5.

More specifically, A4.5N formulation includes the components of Table 3:

TABLE 3
A4.5N (pH 4.5)

| | | |
|---|---|---|
| Sodium acetate buffer | 10 | mM |
| NaCl | 150 | mM |
| Lidocaine | 4 | mg/ml |
| Tetracaine | 2 | mg/ml |

In one embodiment, the candidate formulation (A4.5S) is a solution that includes the following components: lidocaine, tetracaine and sorbitol in sodium acetate buffer. The pH of the reconstituted solution is approximately 3.5.

More specifically, A4.5S formulation includes the components of Table 4:

TABLE 4
A4.5S (pH 4.5)

| Sodium acetate buffer | 10 | mM |
|---|---|---|
| Sorbitol | 5% | |
| Lidocaine | 4 | mg/ml |
| Tetracaine | 2 | mg/ml |

Each of the candidate formulations (herein after referred to as “A3.5N,” “A3.5S,” “A4.5N” or “A4.5S” solution) was studied for its ability to improve the stability of tetracaine and lidocaine. Accordingly, the stability of each anesthetic molecule was monitored over time (i.e., one week, three weeks, four weeks, nine weeks, six months and twenty-four months). The study was repeated and samples were incubated at various temperatures (i.e. 5° C., 25° C., 40° C. and 50° C.). At each time point, samples were analyzed by RP-HPLC.

### Tetracaine Stability

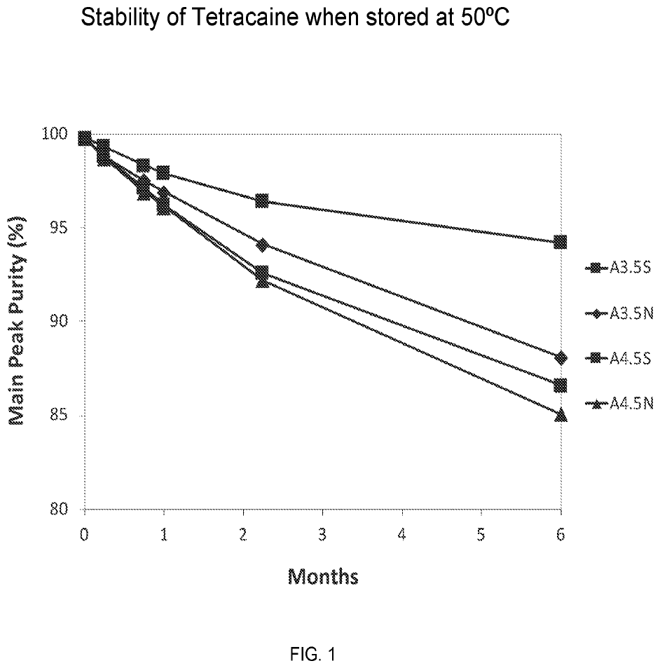

FIG. 1 shows the stability of tetracaine when stored at 50° C. in each of the four candidate solutions (A3.5N, A3.5S, A4.5N and A4.5S). Samples were analyzed by RP-HPCL at each time point (i.e., one week, three weeks, four weeks, nine weeks and six months). The RP-HPLC parameters used were as follows:
Column: Kromasil C18, 4.0×150 mm, part #MH3CLB15, serial #E183536
Mobile Phase A: 0.01M KPi
Mobile Phase B: 100% ACN
Column Temperature: 30° C.
Flow Rate: 1.0 mL/min
Run Time: 28 min
Sample load: 5 μL (2.5 μg)
Absorbance Detection: 254 nm (Lidocaine), 300 nm (Tetracaine detection)

TABLE 5
HPLC parameters

| Time | % Mobile | % Mobile |
|---|---|---|
| (min) | Phase A | Phase B |
| 0 | 100 | 0 |
| 3 | 100 | 0 |
| 18 | 0 | 100 |
| 23 | 0 | 100 |
| 23.1 | 100 | 0 |
| 28 | 100 | 0 |

Each molecule (i.e., tetracaine and lidocaine) produced distinct peaks. Accordingly, the stability was determined by the percentage of the main peak when compared to t=0. Samples were relatively stable for up to three weeks. Thereafter, the instability was apparent from the reduced size of the peak. FIG. 1 shows that A3.5S conferred the greatest stability. Approximately 95% of the molecule remained after incubation for 24 weeks.

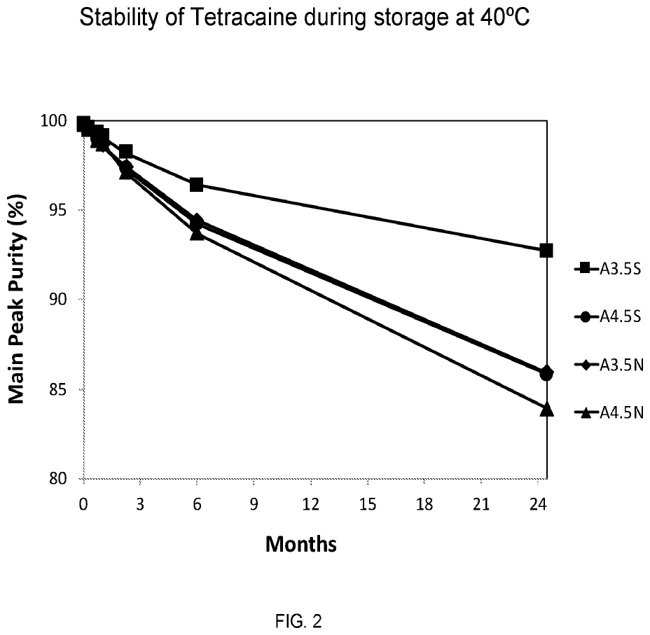

Similarly, FIG. 2 shows the stability of tetracaine when stored at 40° C. in each of the four candidate solutions. Samples were analyzed by HPCL at various time points: one week, three weeks, four weeks, nine weeks, six months and twenty-four months. FIG. 2 also shows that A3.5S conferred the greatest stability. Approximately 93% of the molecule remained after incubation for 2 years.

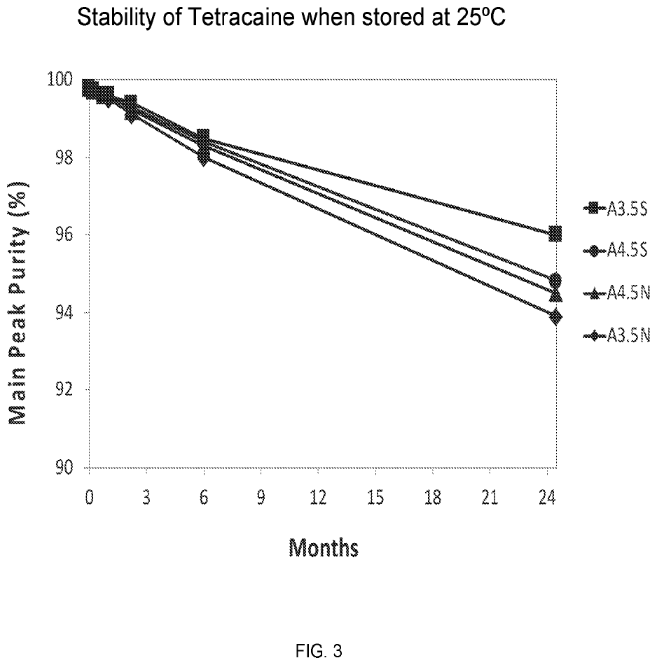

FIG. 3 shows the stability of tetracaine when stored at 25° C. Samples were analyzed by HPCL at the same time points listed above. FIG. 3 also shows that A3.5S conferred the greatest stability. Approximately 96.0% of the molecule remained after incubation for two years.

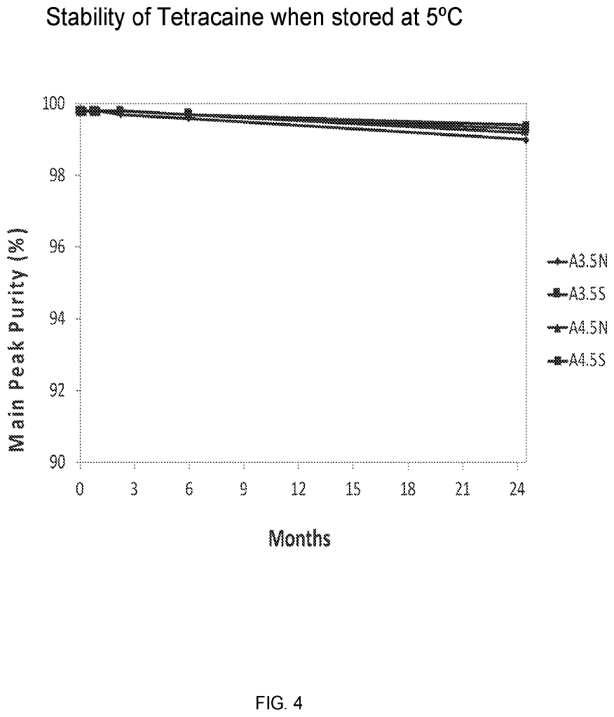

FIG. 4 shows the stability of tetracaine when stored at 5° C. Samples were analyzed by HPCL at the same time points listed above. FIG. 4 shows that each of the solutions conferred approximately the same stability. Tetracaine is relatively stable at low temperatures. Overall, the data demonstrates that tetracaine is most stable in A3.5S formulation. The stability confirming properties of A3.5S formulation are most pronounced at higher temperatures.

### Lidocaine Stability

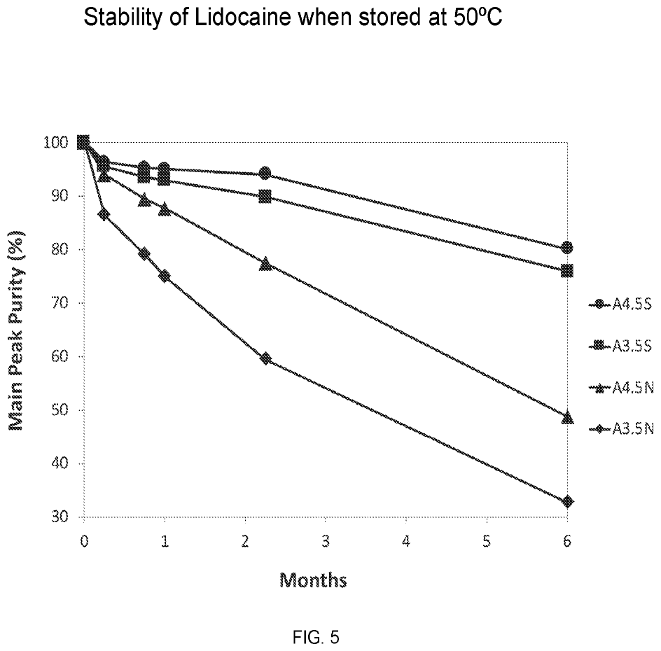

The studies were also used to analyze lidocaine stability. FIG. 5 shows the stability of lidocaine when stored at 50° C. Samples were analyzed by HPCL at the same time points listed above. FIG. 5 shows that formulations containing the non-ionic tonicity modifier sorbitol (A4.5S and A3.5S) conferred the greatest stability. Approximately 85% of the molecule remained after incubation for 24 weeks with sorbitol.

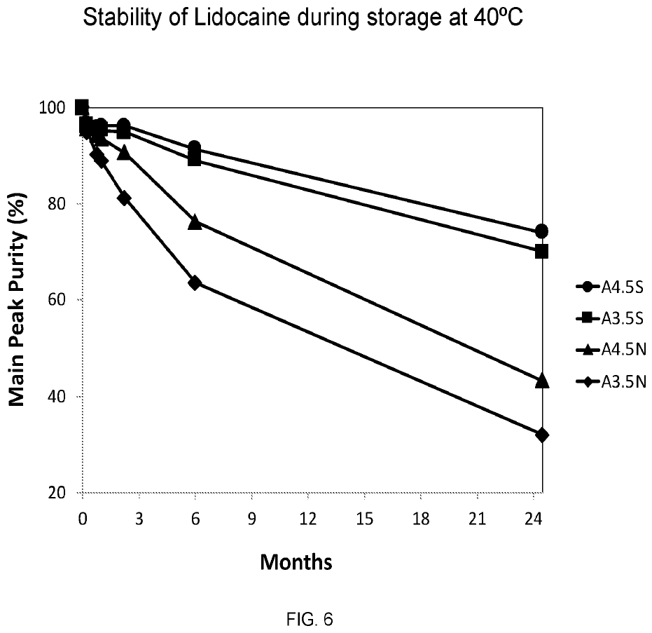

Similarly, FIG. 6 shows the stability of lidocaine when stored at 40° C. Samples were analyzed by HPCL at the same time points listed above. FIG. 6 also shows that formulations containing the non-ionic sorbitol (A4.5S and A3.5S) conferred the greatest stability. Approximately 95% of the molecule remained after incubation for 24 weeks.

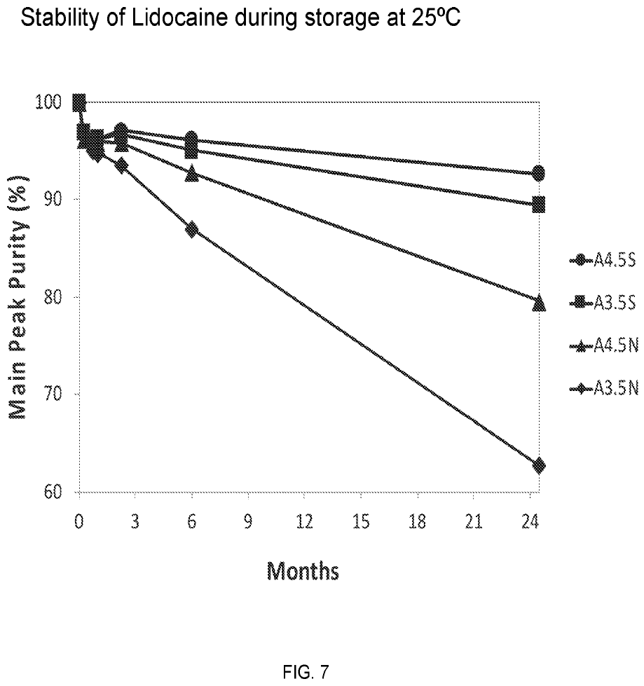

FIG. 7 shows the stability of lidocaine when stored at 25° C. Samples were analyzed by HPCL at the same time points listed above. FIG. 7 also shows that formulations containing the non-ionic sorbitol (A4.5S and A3.5S) conferred the greatest stability. Approximately 90 and 93% of the molecule remained after incubation for two years at pH 3.5 and 4.5, respectively.

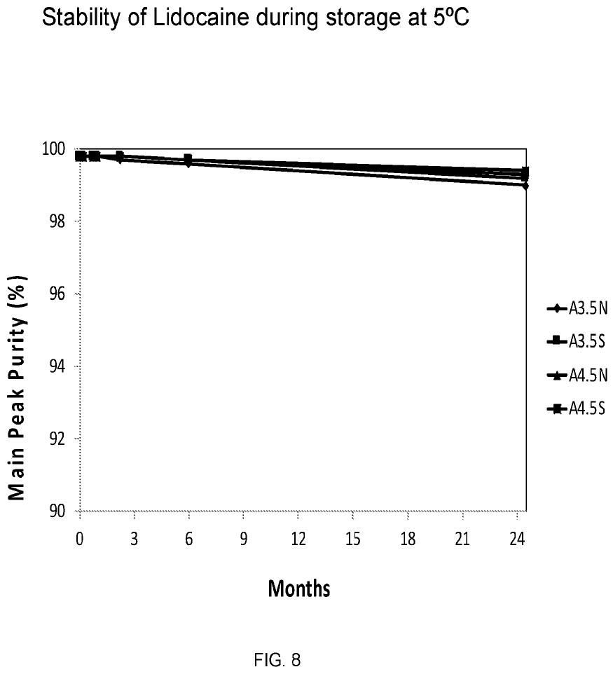

FIG. 8 shows the stability of lidocaine when stored at 5° C. Samples were analyzed by HPCL at the same time points listed above. FIG. 8 shows that each of the solutions conferred approximately the same stability with no significant change in purity during the 24-week storage period.

### Stability in A3.5S Solution

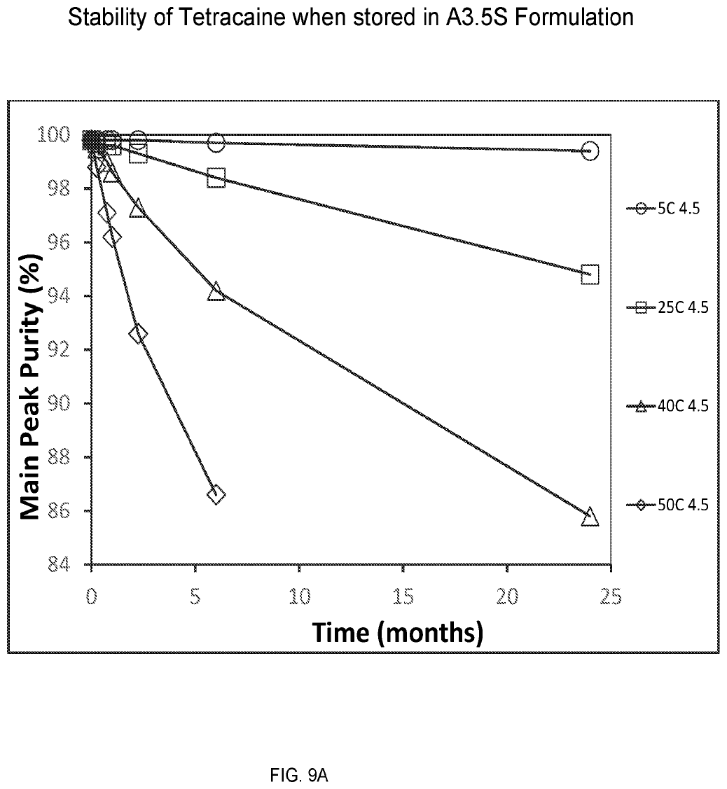

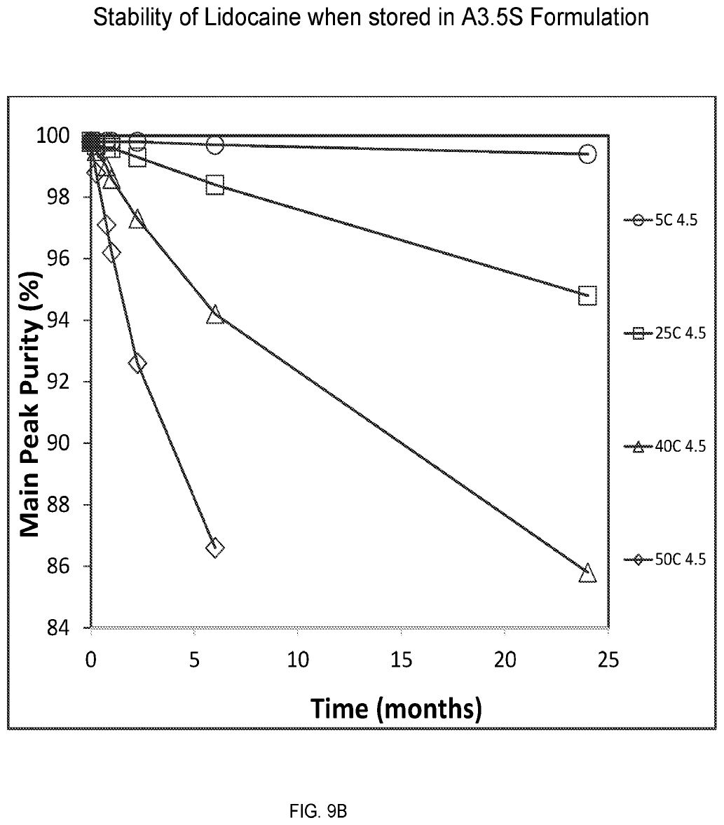

FIG. 9A shows that stability of tetracaine in A3.5S formulation when stored at various temperatures (i.e., 5° C., 25° C., 40° C. and 50° C.). In this solution, the tetracaine is stable for up two years at 5° C. and at 25° C. with purity greater than 95%. Approximately 93% of the molecule was detected after six months at 40° C.

FIG. 9B shows the stability lidocaine in A3.5S formulation when stored at the same temperatures. Lidocaine remained greater than 90% at both 5° C. and 25° C. during two-year storage in the A3.5S formulation. At 40° C., lidocaine could be stored for two months before reaching 95% purity. Degradation was more apparent at 50° C.

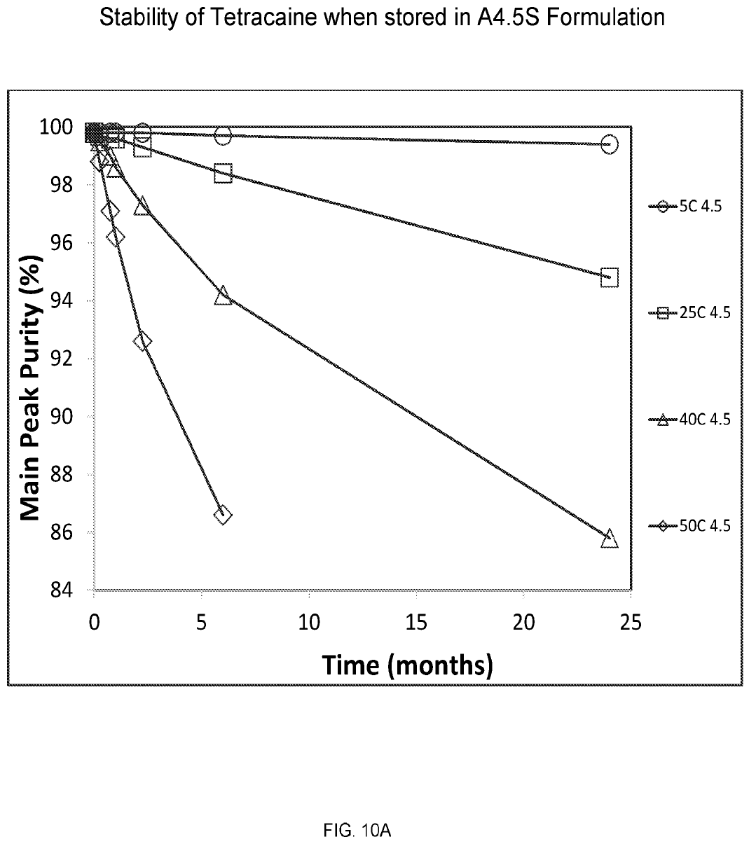

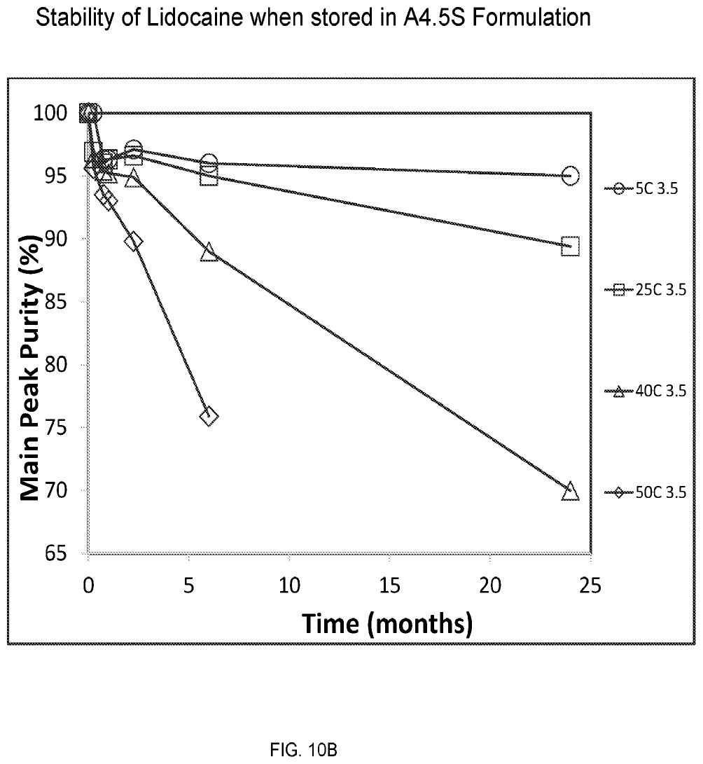

Similarly, FIG. 10A shows that stability of tetracaine in A4.5S formulation when stored up to two years at the various temperatures. In this solution, the tetracaine is generally stable for at temperatures between 5° C. and 25° C. as indicated by greater than 95% purity. It took almost two months at 40° C. and about a month at 50° C. to reach 95% purity.

FIG. 10B shows that stability lidocaine in A4.5S formulation when stored up to two years at various temperatures. For lidocaine, the purity remained above 93% at 5° C. and 25° C. during storage for two years at 5-25° C. It took about six months and two months for the purity to decline to 90% during storage at 40° C. and 50° C., respectively.

The studies demonstrate that sorbitol formulations showed better stability than NaCl formulations. For tetracaine, greater stability was demonstrated at pH 3.5 than at pH 4.5. Tetracaine is only stable at pH 3.5 in the presence of non-ionic tonicity modifier like sorbitol (A3.5S). Lidocaine is not stable at low pH (around 3.5) in normal saline but becomes stable when the tonicity modifier is replaced with a non-ionic tonicity modifier. Thus, A3.5S is a unique formulation in which both tetracaine and lidocaine are stable in liquid state.

Similarly, both tetracaine and lidocaine showed sufficient stability when stored at pH 4.5 in the sorbitol formulation. While the stability of tetracaine is slightly inferior at pH 4.5 than at pH 3.5, lidocaine showed slightly better stability at pH 4.5 than at pH 3.5. Both products show sufficient stability for two years at storage temperatures of 5 and 25° C.

In some aspects, the compositions and methods described herein address the problem of providing stable anesthetic molecules, while maintaining administration practicability and stability. It has been found that the addition of a non-ionic tonicity modifier can improve the stability of an anesthetic in solution. Thus, one embodiment provides a stable pharmaceutical formulation comprising an anesthetic and one or more non-ionic tonicity modifiers. In one aspect, a tonicity modifier is present at a concentration of at least about 0.1% (w/v), or alternatively at least about 0.01%, 0.02%, 0.05%, 0.075%, 0.2%, 0.3%, 0.4%, 0.5%, 0.6%, 0.7%, 0.8%, 0.9%, 1%, 1.5%, 1.75%, 2%, 2.25%, 2.5%, 2.75%, 3%, 3.25%, 3.5%, 3.75%, 4%, 4.25%, 4.5%, 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, or more (w/v).

In a further aspect, a tonicity modifier is present at a concentration of about 0.1% (w/v) to about 10%, or alternatively at about 0.01% to about 10%, 0.02% to about 10%, 0.05% to about 10%, 0.075% to about 10%, 0.2% to about 10%, 0.3% to about 10%, 0.4% to about 10%, 0.5% to about 10%, 0.6% to about 10%, 0.7% to about 10%, 0.8% to about 10%, 0.9% to about 10%, 1% to about 10%, 1.5% to about 10%, 1.75% to about 10%, 2% to about 10%, 2.25% to about 10%, 2.5% to about 10%, 2.75% to about 25%, 3% to about 25%, 3.25% to about 10%, 3.5% to about 10%, 3.75% to about 10%, 4% to about 10%, 4.25% to about 10%, 4.5% to about 10%, 4.75% to about 10%, 5% to about 10%, (w/v).

In an embodiment, an anesthetic can be said to “remain stable” in a pharmaceutical formulation, if, for example, between about 50% and about 100% of the molecule, such as lidocaine or tetracaine, remains after a given time; or alternatively between about 60% and about 100% remains, or alternatively between about 70% and about 100% remains, or alternatively between about 80% and about 100% remains, or alternatively between about 90% and about 100% remains, e.g., as detected by RP-HPLC. The given time can be, for example, 24 months.

In any of the above embodiments of the formulations containing combinations of anesthetic(s), the formulations can have a pH that is from about 3 to about 5. Alternatively, in a further embodiment, the pH is from about 2 to about 4, from about 3 to about 4, or from about 2.5 to about 3.5. In an embodiment, the pH is at least about 2, 2.25, 2.5, 2.75, 3, 3.25, 3.5, 3.75, 4, 4.25, 4.5. In a further embodiment, the pH of the formulation is in the range of about 2 to about 4, about 2.5 to about 4.5, about 3.0 to about 5.0, about 2 to about 3, about 2.5 to about 4.0, about 2.5 to about 3.5, about 3.0 to about 4.0.

By virtue of the stabilizing effect of the non-ionic tonicity modifier, the compositions and methods described herein permit the preparation of stable anesthetic formulations that can be routinely processed for pharmaceutical manufacturing including pumping, filtration, ultrafiltration, diafiltration, freeze/thawing, filling, lyophilization, sterilization, etc.

By virtue of the stabilizing effect of the tonicity modifier, the compositions and methods described herein permit the preparation of stable anesthetic formulations that can be exposed to routine pharmaceutical operation environment including, subzero temperature, refrigeration, elevated temperature, transportation, storage, ambient temperature storage, exposure to light, and administration devices and conditions.

### EXAMPLES

The compositions and methods described herein will be further understood by reference to the following examples, which are intended to be purely exemplary. The compositions and methods described herein are not limited in scope by the exemplified embodiments, which are intended as illustrations of single aspects only. Any methods that are functionally equivalent are within the scope of the invention. Various modifications of the compositions and methods described herein in addition to those expressly described herein will become apparent to those skilled in the art from the foregoing description and accompanying figures. Such modifications fall within the scope of the invention.

### Example 1

### Stability in A3.5N Solution

In the following example, the formulation (herein after referred to as “A3.5N” or “A3.5N solution”) contains, 150 mM NaCl in 10 mM sodium acetate buffer at pH 3.5. Tetracaine stability was studied by adding 2 mg/mL of tetracaine to A3.5N solution. Similarly, lidocaine stability was studied by adding 4 mg/mL of lidocaine to A3.5N solution.

In this study, the solutions were separated into four vials. The vials were incubated at each of 5° C., 25° C., 40° C. and 50° C. Samples from each vial were examined at one week, three weeks, four weeks, nine weeks, six months and twenty-four months. Samples were then analyzed by RP-HPLC method using the parameters described above.

Each molecule (i.e. tetracaine and lidocaine) produced single distinct peaks in their pure forms. Accordingly, the stability was determined by the percentage of the main peak when compared to t=0. Table 6 and 7 show the results when each of the anesthetics was incubated in A3.5N solution.

TABLE 6
Experimental Results:
A3.5N - Tetracaine Purity (%)

| | 5° C. | 25° C. | 40° C. | 50° C. |
|---|---|---|---|---|
| Time = 0 | 99.8 | — | — | — |
| one week | — | 99.7 | 99.5 | 98.8 |
| three weeks | 99.8 | 99.6 | 98.9 | 97.5 |
| four weeks | 99.8 | 99.5 | 98.6 | 96.9 |
| nine weeks | 99.7 | 99.1 | 97.4 | 94.1 |
| six months | 99.6 | 98.5 | 94.4 | 88.1 |
| Two years | 99.0 | 93.9 | 85.9 | N/A |

TABLE 7
Experimental Results:
A3.5N - Lidocaine Purity (%)

| | 5° C. | 25° C. | 40° C. | 50° C. |
|---|---|---|---|---|
| Time = 0 | 100 | — | — | — |
| one week | — | 96.6 | 94.8 | 86.6 |
| three weeks | 96.0 | 95.0 | 90.2 | 79.1 |
| four weeks | 94.6 | 94.7 | 88.9 | 74.9 |
| nine weeks | 97.1 | 93.5 | 81.1 | 59.5 |
| six months | 95.5 | 87.0 | 63.5 | 32.7 |
| Two years | 92.1 | 62.7 | 31.9 | N/A |

### Example 2

### Stability in A3.5S Solution

In the following example, the formulation (herein after referred to as “A3.55” or “A3.55 solution”) contains 4 mg/mL of lidocaine, 2 mg/mL of tetracaine, 5% sorbitol in 10 mM sodium acetate buffer at pH 3.5 (Table 8-9).

TABLE 8
Experimental Results:
A3.5S - Tetracaine Purity (%)

| | 5° C. | 25° C. | 40° C. | 50° C. |
|---|---|---|---|---|
| Time = 0 | 99.8 | — | — | — |
| one week | — | 99.7 | 99.5 | 98.8 |
| three weeks | 99.8 | 99.6 | 99.3 | 97.5 |
| four weeks | 99.8 | 99.5 | 98.6 | 97.9 |
| nine weeks | 97.1 | 96.6 | 94.9 | 89.8 |
| six months | 99.7 | 98.5 | 96.4 | 94.2 |
| Two years | 99.2 | 96.0 | 92.7 | N/A |

TABLE 9
Experimental Results:
A3.5S - Lidocaine Purity (%)

| | 5° C. | 25° C. | 40° C. | 50° C. |
|---|---|---|---|---|
| Time = 0 | 100 | — | — | — |
| one week | — | 96.6 | 94.8 | 86.4 |
| three weeks | 96.3 | 96.0 | 95.4 | 93.5 |
| four weeks | 96.3 | 96.3 | 95.2 | 93.0 |
| nine weeks | 97.1 | 96.6 | 94.9 | 89.8 |
| six months | 96.0 | 95.0 | 89.0 | 75.9 |
| Two years | 95.0 | 89.4 | 70.0 | N/A |

### Example 3

### Stability in A4.5N Solution

In the following example, the formulation (herein after referred to as “A4.5N” or “A4.5N solution”) contains 4 mg/mL of lidocaine, 2 mg/mL of tetracaine, 150 mM NaCl in 10 mM sodium acetate buffer at pH 4.5 (Table 10-11).

TABLE 10
Experimental Results:
A4.5N - Tetracaine Purity (%)

| | 5° C. | 25° C. | 40° C. | 50° C. |
|---|---|---|---|---|
| Time = 0 | 99.8 | — | — | — |
| one week | — | 99.7 | 99.5 | 98.7 |
| three weeks | 99.8 | 99.6 | 98.9 | 96.9 |
| four weeks | 99.8 | 99.6 | 98.7 | 96.1 |
| nine weeks | 99.8 | 99.2 | 97.1 | 92.2 |
| six months | 99.7 | 98.3 | 93.7 | 85.1 |
| Two years | 99.3 | 94.5 | 83.9 | N/A |

TABLE 11
Experimental Results:
A4.5N - Lidocaine Purity (%)

| | 5° C. | 25° C. | 40° C. | 50° C. |
|---|---|---|---|---|
| Time = 0 | 100 | — | — | — |
| one week | — | 96.2 | 95.6 | 93.9 |
| three weeks | 96.0 | 95.9 | 94.3 | 89.4 |
| four weeks | 96.4 | 95.9 | 93.6 | 87.6 |
| nine weeks | 97.1 | 95.8 | 90.6 | 77.4 |
| six months | 96.1 | 92.8 | 76.2 | 48.7 |
| Two years | 94.7 | 79.6 | 43.2 | N/A |

### Example 4

### Stability in A4.5S Solution

In the following example, the formulation (herein after referred to as “A4.5S” or “A4.5S solution”) contains 4 mg/mL of lidocaine, 2 mg/mL of tetracaine, 5% (w/w) sorbitol in 10 mM sodium acetate buffer at pH 4.5 (Table 12-13).

TABLE 12
Experimental Results:
A4.5S - Tetracaine Purity (%)

| | 5° C. | 25° C. | 40° C. | 50° C. |
|---|---|---|---|---|
| Time = 0 | 99.8 | — | — | — |
| one week | — | 99.7 | 99.5 | 98.8 |
| three weeks | 99.8 | 99.6 | 99.0 | 97.1 |
| four weeks | 99.8 | 99.6 | 98.7 | 96.1 |
| nine weeks | 99.8 | 99.2 | 97.1 | 92.2 |
| six months | 99.8 | 99.6 | 99.0 | 97.1 |
| Two years | 99.4 | 94.8 | 85.8 | N/A |

TABLE 13
Experimental Results:
A4.5S - Lidocaine Purity (%)

| | 5° C. | 25° C. | 40° C. | 50° C. |
|---|---|---|---|---|
| Time = 0 | 100 | — | — | — |
| one week | — | 96.6 | 94.8 | 96.3 |
| three weeks | 96.2 | 96.0 | 95.9 | 95.3 |
| four weeks | 96.5 | 96.3 | 96.1 | 95.0 |
| nine weeks | 97.2 | 97.1 | 96.1 | 94.0 |
| six months | 96.4 | 96.1 | 91.3 | 80.1 |
| Two years | 96.0 | 92.6 | 74.0 | N/A |

The results demonstrate that the stability of lidocaine is better at pH 4.5 than at pH 3.5 in normal saline (150 mM NaCl). The stability of lidocaine is further enhanced by replacing normal saline with 5% sorbitol. Lidocaine is stable both at pH 4.5 and pH 3.5 in the presence of 5% sorbitol. The stability of tetracaine is better at pH 3.5 when formulated with 5% sorbitol. Comparable stability was observed at both pH 3.5 and 4.5 when 5% sorbitol was used as tonicity modifier. Both tetracaine and lidocaine are stable in liquid state when formulated at pH 3.5 of pH 4.5 with 5% sorbitol as tonicity modifier.

The formulations can be used for other anesthetic agents such as procaine, amethocaine, cocaine, benzocaine, tetracaine, lidocaine, prilocaine, bupivicaine, levobupivacaine, ropivacaine, mepivacaine, dibucaine and/or etidocaine.

### Example 5

### Use of Tetracaine in Solution to Alleviate Pain

Tetracaine is a local anesthetic used to numb the eyes, nose, or throat. In this example, it is applied to the skin before starting an intravenous (IV) or giving an epidural shot to decrease pain from the procedure. Because injections of local anesthetics are painful, a topical anesthetic is often used to numb the target area.

In this example, the tetracaine is provided in a stabilizing solution that contains 2 mg/mL of tetracaine, in 10 mM sodium acetate buffer with 5% sorbitol as tonicity modifier at pH 3.5-4.5. The solution can also be provided in an ointment, gel or spray by addition of emulsifying agents known in the art.

The target area is first cleaned and prepped. Thereafter, the tetracaine solution is applied. The patient can experience numbness within minutes. Thereafter, the healthcare provider can proceed with the intravenous or epidural. The tetracaine solution can also be used to numb the eyes, nose, or throat.

### Example 6

### Use of Stable LET Solution to Alleviate Pain

Currently lidocaine is stored and shipped in isotonic solution containing normal saline with the pH 3.5-4.5. Because tetracaine is not stable in this formulation, the combination liquid product is not available. For this reason, LET is conventionally available as a kit with four lyophilized ingredients: lidocaine hydrochloride, epinephrine bitartrate, tetracaine hydrochloride and sodium metabisulfite. The ingredients must be resuspended in sterile solution prior to use.

In this example, the LET is provided in a stabilizing solution that contains 2 mg/mL of tetracaine, 4 mg/ml lidocaine in 10 mM sodium acetate buffer with 5% sorbitol as a tonicity modifier at pH 3.5-4.5. The LET remains stable in solution at refrigerated or ambient temperature. Lyophilization is not necessary. The solution can also be provided in an ointment, gel or spray by addition of emulsifying agents known in the art.

LET can be used to anesthetize the skin before intramuscular injections, venipuncture, and simple skin procedures such as curettage or biopsy. To be fully effective, LET can be applied at least 90 minutes before the procedure. The target area is first cleaned and prepped. Thereafter, the tetracaine solution is applied. The patient can experience numbness within minutes. Thereafter, the healthcare provider can conduct the procedure.

As provided above, in one embodiment, sorbitol can be present at a concentration of at least about 0.1% (w/v), or alternatively at least about at about 0.01% to about 10%, 0.02% to about 10%, 0.05% to about 10%, 0.075% to about 10%, 0.2% to about 10%, 0.3% to about 10%, 0.4% to about 10%, 0.5% to about 10%, 0.6% to about 10%, 0.7% to about 10%, 0.8% to about 10%, 0.9% to about 10%, 1% to about 10%, 1.5% to about 10%, 1.75% to about 10%, 2% to about 10%, 2.25% to about 10%, 2.5% to about 10%, 2.75% to about 10%, 3% to about 10%, 3.25% to about 10%, 3.5% to about 10%, 3.75% to about 10%, 4% to about 10%, 4.25% to about 10%, 4.5% to about 10%, 4.75% to about 10%, 5% to about 10%, 5.25% to about 10%, 5.5% to about 10%, 5.75% to about 10%, 6% to about 10%, 6.25% to about 10%, 6.5% to about 10%, 6.75% to about 10%, 7% to about 10%, 7.25% to about 10%, 7.5% to about 10%, 7.75% to about 10%, 8% to about 10%, 8.25% to about 10%, 8.5% to about 10%, 8.75% to about 10%, 9% to about 10%, 9.25% to about 10%, 9.5% to about 10%, 9.75% to about 10%, Still further, in an additional embodiment, sorbitol can be present at a concentration of between about 0.1 mg/ml to about 10 mg/ml, or between about 1 mg/ml to about 20 mg/ml, or between about 0.5 mg/ml to about 10 mg/ml, or between about 2 mg/ml to about 10 mg/ml. In an embodiment, each amino acid may be present at a concentration of at least about 0.01 mg/ml, 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, or any combination thereof.

In one embodiment, any formulation as described herein is in a liquid form. In another embodiment, any formulation as described herein is provided in a container closure system that is a prefilled syringe.

In an embodiment, a pharmaceutical formulation is comprised of an anesthetic molecule as an active biological agent and a soluble formulation within which the active agent is dissolved. In some embodiments, the soluble formulation is a formulation as described herein to further include one or more excipients, such as buffers, tonicity modifiers, bulking agents, metal ions, chelating agents, surfactants, stabilizers, polymers, salts, carbohydrates, proteins etc. As used herein, the term “excipient” refers to an inert substance which is commonly used as a diluent, vehicle, preservative, binder, or stabilizing agent for drugs and includes, but is not limited to, proteins (e.g., serum albumin, etc.), fatty acids and phospholipids (e.g., alkyl sulfonates, caprylate, etc.), surfactants (e.g., polysorbate, poloxamer, nonionic surfactant, etc.), and carbohydrates (e.g., mannitol, sorbitol, sucrose, maltose, trehalose, etc.). Also see Remington's Pharmaceutical Sciences (by Joseph P. Remington, 18th ed., Mack Publishing Co., Easton, Pa.) and Handbook of Pharmaceutical Excipients (by Raymond C. Rowe, 5th ed., APhA Publications, Washington, DC) which are hereby incorporated in its entirety. The excipients may impart a beneficial physical property to the formulation, such as increased product stability and increased product solubility.

In an embodiment, liquid suspensions can be formulated by suspending a therapeutic compound disclosed herein in a mixture with excipients suitable for the manufacture of aqueous suspensions. In an embodiment, such excipients are suspending agents, for example, sodium carboxymethylcellulose, methylcellulose, hydroxypropylmethylcellulose, sodium alginate, pectin, polyvinyl pyrrolidone, polyvinyl alcohol, natural gum, agar, gum tragacanth and gum acacia; dispersing or wetting agents may be a naturally occurring phosphatide, for example lecithin, or condensation products of an alkylene oxide with fatty acids, for example, polyoxyethylene stearate, or condensation products of ethylene oxide with long-chain aliphatic alcohols, for example, heptadecaethyleneoxycetanol, or condensation products of ethylene oxide with partial esters derived from fatty acids, for example, without limitation, polyoxyethylene sorbitan monooleate.

Particular excipients as approved for U.S. Food and Drug regulatory purposes can be found at the FDA Inactive Ingredient Database. Many useful excipients are well known in the art and can be found described in, for example, Banga, A. K., Therapeutic Peptides and Proteins, Formulation, Processing and Delivery Systems, (2d Ed 2006, CRC Press), Chapter 4, section 4.4, Pharmaceutical excipients in formulations (at pages 104-116). Any one or more of any other excipients or others may be included in any formulation as described herein. Similarly, in an embodiment, at least one excipient can confer more than one of the functions onto a formulation. Alternatively, in another embodiment, two or more excipients can be included in a formulation to perform more than one of the above or other functions. For example, an excipient can, without limitation, be included as a component in a formulation to change, adjust or optimize the osmolality of the formulation, thereby acting as a tonicity modifier.

In various embodiments, a formulation can include, without limitation, combinations of bioactive agents (such as an anesthetic as described herein) in the formulation. For example, a formulation as described herein can include a single bioactive agent for treatment of one or more conditions, including without limitation, disease. A formulation as described herein also can include, in an embodiment, without limitation, two or more different bioactive agents for a single or multiple conditions. Use of multiple bioactive agents in a formulation can be directed to, for example, the same or different indications. Similarly, in another embodiment, multiple bioactive agents can be used in a formulation to treat, for example, both a pathological condition and one or more side effects caused by the primary treatment. In a further embodiment, multiple bioactive agents also can be included, without limitation, in a formulation as described herein to accomplish different medical purposes including, for example, simultaneous treatment and monitoring of the progression of the pathological condition. In an additional embodiment, multiple, concurrent therapies such as those exemplified herein as well as other combinations well known in the art are particularly useful for patient compliance because a single formulation can be sufficient for some or all suggested treatments and/or diagnosis. Those skilled in the art will know those bioactive agents that can be admixed for a wide range of combination therapies. Similarly, in various embodiments, a formulation can be used with a small molecule drug and combinations of one or more bioactive agents s together with one or more small molecule pharmaceuticals. Therefore, in various embodiments a formulation is provided containing 1, 2, 3, 4, 5 or 6 or more different bioactive agents, as well as, for one or more bioactive agents combined with one or more small molecule pharmaceuticals.

In various embodiments, a formulation can include, one or more preservatives and/or additives known in the art. Similarly, a formulation can further be formulated, without limitation, into any of various known delivery formulations. For example, in an embodiment, a formulation can include, surfactants, adjuvant, biodegradable polymers, hydrogels, etc., such optional components, their chemical and functional characteristics are known in the art. Similarly known in the art are formulations that facilitate rapid, sustained or delayed release of the bioactive agents after administration. A formulation as described can be produced to include these or other formulation components known in the art.

Once a formulation is prepared as described herein, stability of the one or more active pharmaceutical agents contained within the formulation can be assessed using methods known in the art. Several methods are exemplified herein in the Examples and include various HPLC methods. Other methods can comprise any of a variety of functional assays including, for example, binding activity, other biochemical activity and/or physiological activity can be assessed at two or more different time points to determine the stability of the active agents in a formulation as described herein. A formulation can, in general, be prepared according to pharmaceutical standards and using pharmaceutical grade reagents. Similarly, a formulation can be prepared using sterile reagents in a sterile manufacturing environment or sterilized following preparation. Sterile injectable solutions can be prepared using known procedures in the art including, for example, by incorporating one or more bioactive agents s in the required amount in a glutamic acid buffer or excipient with one or a combination of formulation components described herein followed by sterilization microfiltration. In various embodiments, sterile powders for the preparation of sterile injectable solutions can include, for example, vacuum drying and freeze-drying (lyophilization). Such drying methods will yield a powder of the one or more bioactive agents s together with any additional desired components from a previously sterile-filtered solution thereof.

In an embodiment, further representative excipients include, without limitation, inorganic salt or buffers such as citric acid, sodium chloride, magnesium chloride, manganese chloride, potassium chloride, sodium sulfate, ammonium sulfate, potassium nitrate, sodium phosphate monobasic, sodium phosphate dibasic, and combinations thereof.

In an embodiment, further representative excipients include, without limitation polypeptides, e.g., proteins or peptides, such as serum albumin, cytokines, immunoglobulins, enzymes, gelatin, plasma proteins, etc.

In an embodiment, formulations suitable for nasal administration, wherein the carrier is a solid, include a coarse powder having a particle size, for example, in the range of about 0.2 to about 500 microns. In a further embodiment, particle size is, at least, 0.2 microns, 0.5 microns, 1 microns, 5 microns, 10 microns, 20 microns, 30 microns, 40 microns, 50 microns, 60 microns, 70 microns, 80 microns, 90 microns, 100 microns, 110 microns, 120 microns, 130 microns, 140 microns, 150 microns, 160 microns, 170 microns, 180 microns, 190 microns, 200 microns, 210 microns, 220 microns, 230 microns, 240 microns, 250 microns, 260 microns, 270 microns, 280 microns, 290 microns, 300 microns, 310 microns, 320 microns, 330 microns, 340 microns, 350 microns, 360 microns, 370 microns, 380 microns, 390 microns, 400 microns, 410 microns, 420 microns, 430 microns, 440 microns, 450 microns, 460 microns, 470 microns, 480 microns, 490 microns, 500 microns, 600 microns, 700 microns, 800 microns, 900 microns, 1000 microns. In an additional embodiment, such a formulation is typically administered, without limitation, by rapid inhalation through the nasal passage, for example, from a container of the powder held in proximity to the nose. In an additional embodiment, such a formulation is typically administered, by rapid inhalation through the mouth, for example, from a container of the powder held in proximity to the mouth. In an embodiment, a formulation for nasal delivery can be in the form of a liquid, e.g., a nasal spray or nasal drops.

In an embodiment, aerosolizable formulations for inhalation can be in dry powder form (e.g., suitable for administration by a dry powder inhaler), or, alternatively, may be in liquid form, e.g., for use in a nebulizer. In an embodiment, nebulizers for delivering an aerosolized solution include the AERx™ (Aradigm), the Ultravent® (Mallinkrodt), and the Acorn II® (Marquest Medical Products). In an embodiment, a composition of the invention can also be delivered using a pressurized, metered dose inhaler (MDI), e.g., the Ventolin® metered dose inhaler, containing a solution or suspension of a combination of drugs as described herein in a pharmaceutically inert liquid propellant, for example, a chlorofluorocarbon or fluorocarbon.

In an embodiment, formulations suitable for parenteral administration include aqueous and non-aqueous isotonic sterile solutions suitable for injection, as well as aqueous and non-aqueous sterile suspensions. In an embodiment, parenteral formulations of the invention are optionally contained in unit-dose or multi-dose sealed containers, for example, ampoules and vials, and can be stored in a freeze-dried (lyophilized) condition requiring only the addition of the sterile liquid carrier, for example, water for injections, immediately prior to use.

The composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data. In various embodiments, the bioactive agents in formulations described herein can, without limitation, be administered to patients throughout an extended time period, such as chronic administration for a chronic condition.

The formulation described in this specification may also comprise more than one therapeutic anesthetic molecules as desired for the particular indication being treated, preferably those with complementary activities that do not adversely affect the other anesthetics. The formulations to be used for in vivo administration can be sterile. This can be accomplished, for instance, without limitation, by filtration through sterile filtration membranes, prior to, or following, preparation of the formulation or other methods known in the art, including without limitation, pasteurization.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

Injection devices include pen injectors, auto injectors, safety syringes, injection pumps, infusion pumps, glass prefilled syringes, plastic prefilled syringes and needle free injectors syringes may be prefilled with liquid, or may be dual chambered, for example, for use with lyophilized material. An example of a syringe for such use is the Lyo-Ject™, a dual-chamber pre-filled lyosyringe available from Vetter GmbH, Ravensburg, Germany. Another example is the LyoTip which is a prefilled syringe designed to conveniently deliver lyophilized formulations available from LyoTip, Inc., Camarillo, Calif., U.S.A. Administration by injection may be, without limitation intravenous, intramuscular, intraperitoneal, or subcutaneous, as appropriate. Administrations by non-injection route may be, without limitation, nasal, oral, cocular, dermal, or pulmonary, as appropriate.

In certain embodiments, kits can comprise, without limitation, one or more single or multi-chambered syringes (e.g., liquid syringes and lyosyringes) for administering one or more formulations described herein. In various embodiments, the kit can comprise formulation components for parenteral, subcutaneous, intramuscular or IV administration, sealed in a vial under partial vacuum in a form ready for loading into a syringe and administration to a subject. In this regard, the composition can be disposed therein under partial vacuum. In all of these embodiments and others, the kits can contain one or more vials in accordance with any of the foregoing, wherein each vial contains a single unit dose for administration to a subject.

The kits can comprise lyophilates, disposed as herein, that upon reconstitution provide compositions in accordance therewith. In various embodiment the kits can contain a lyophilate and a sterile diluent for reconstituting the lyophilate. The reconstituted solution can include the disclosed tonicity modifier (e.g. sorbitol) at a low pH (e.g. 3.5 or 4.5) to improve its stability.

Imaging components can optionally be included and the packaging also can include written or web-accessible instructions for using the formulation. A container can include, for example, a vial, bottle, syringe, pre-filled syringe or any of a variety of formats well known in the art for multi-dispenser packaging.

Also described herein, are methods for treating a subject in need of therapy, comprising administering to the subject an effective amount of a formulation as described herein. The therapeutically effective amount or dose of a formulation will depend on the disease or condition of the subject and actual clinical setting.

In an embodiment, a formulation as described herein can be administered by any suitable route, specifically by parental (including subcutaneous, intramuscular, intravenous, intradermal, and other local area) administration. It will also be appreciated that the preferred route will vary with the condition and age of the recipient, and the disease being treated. Methods of determining the most effective means and dosage of administration are known to those of skill in the art and will vary, without limitation, with the composition used for therapy, the purpose of the therapy, and the subject being treated. Single or multiple administrations can be carried out, without limitation, the dose level and pattern being selected by the treating physician. Suitable dosage formulations and methods of administering the agents are known in the art.

The formulations as described herein can be used in the manufacture of medicaments and for the treatment of humans and other animals by administration in accordance with conventional procedures.

The general process for developing a formulation is discussed below. The process can be divided into three parts: Preformulation Characterization, High Throughput Screening and Long-Term Stability Confirmation.

Preformulation characterization studies generally are designed to understand pharmaceutically significant physicochemical properties of the formulant, such as stability when exposed to common stresses, developing assays for degradation products and other measures of stability, determining if a lyophilized or liquid formulation will be better for initial clinical studies, and developing a final research protocol. Preformulation characterization generally involves physiochemical characterization, stability assay development and stress studies to identify formulation and stability problems and facilitate optimization studies.

High throughput screening typically is used to test a large number of possible formulations to identify a limited number of candidates for development. Most degradation reactions of anesthetics to evaluate stability, e.g., chemical degradations or recovery loss, can be accelerated by exposing to various stresses, e.g., increasing the storage temperature. A high throughput screening protocol can be developed based on critical issues observed at the preformulation characterization. Forced degradation may be used to expedite the relevant degradation pathway(s). Effective stabilizers or their combinations, e.g., tonicity modifiers, are identified by the high throughput screening.

A small number of promising formulations can be selected and tested for long term stability. For example, samples formulated with the selected stabilizer(s) can be prepared in appropriate container/closure system and incubated at appropriate storage conditions or expose other relevant stresses, and characterized at appropriate time points which can last weeks, months or years. Various analytical methods are used to evaluate the integrity of the anesthetic during storage and/or or other relevant stresses. The stabilizers effective under forced degradation studies should be effective in stabilizing the molecule under real life handling, storage, and transportation conditions.

Methods herein described can be useful for carrying out any one or all three stages of the development process, but, are particularly useful for systematically screening the components most likely to provide the desired formulation characteristics and long term stability.

Compositions in accordance with embodiments described herein have desirable properties, such as desirable solubility, viscosity, syringeabilty and stability.

In an embodiment, the formulation includes a non-ionic tonicity modifier. The tonicity modifier can at a concentration of about 0.01%, 0.02%, 0.05%, 0.075%, 0.2%, 0.3%, 0.4%, 0.5%, 0.6%, 0.7%, 0.8%, 0.9%, 1%, 1.5%, 1.75%, 2%, 2.25%, 2.5%, 2.75%, 3%, 3.25%, 3.5%, 3.75%, 4%, 4.25%, 4.5%, 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, or more (w/v). In an embodiment, the tonicity modifier is sorbitol. In an embodiment, the pharmaceutical formulation comprises two or more different tonicity modifiers.

In an embodiment, the pH of the formulation is at least about 2.0, 2.5, 3.0, 3.5, 3.75, 4, 4.25, 4.5, 4.75, 5.

In an embodiment, the pH of the formulation is from about 2 to about 6, about 3 to about 5, about 3.5 to about 4.5 or about 3 to 5.

The formulations described herein can be used with other anesthetic agents including, for example, desflurane, enflurane, halothane, isoflurane, methoxyflurane, sevoflurane, xenon, a barbiturate, a benzodiazepine, etomidate, ketamine, propofol, alfentanil, fentanyl, remifentanil, sufentanil, buprenorphine, butorphanol, diamorphine, hydromorphone, levorphanol, pethidine (meperidine), methadone, morphine, nalbuphine, oxycodone, oxymorphone or pentazocine

In an embodiment, the formulation also includes one or more amino acids at a concentration of at about 0.01%, 0.02%, 0.05%, 0.075%, 0.2%, 0.3%, 0.4%, 0.5%, 0.6%, 0.7%, 0.8%, 0.9%, 1%, 1.5%, 1.75%, 2%, 2.25%, 2.5%, 2.75%, 3%, 3.25%, 3.5%, 3.75%, 4%, 4.25%, 4.5%, 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, or more (w/w).

In an embodiment, the concentration of an amino acid is about 0.1% (w/v) to about 10%, or alternatively at about 0.01% to about 10%, 0.02% to about 10%, 0.05% to about 10%, 0.075% to about 10%, 0.2% to about 10%, 0.3% to about 10%, 0.4% to about 10%, 0.5% to about 10%, 0.6% to about 10%, 0.7% to about 10%, 0.8% to about 10%, 0.9% to about 10%, 1% to about 10%, 1.5% to about 10%, 1.75% to about 10%, 2% to about 10%, 2.25% to about 10%, 2.5% to about 10%, 2.75% to about 10%, 3% to about 10%, 3.25% to about 10%, 3.5% to about 10%, 3.75% to about 10%, 4% to about 10%, 4.25% to about 10%, 4.5% to about 10%, 4.75% to about 10%, 5% to about 10%, 5.25% to about 10%, 5.5% to about 10%, 5.75% to about 10%, 6% to about 10%, 6.25% to about 10%, 6.5% to about 10%, 6.75% to about 10%, 7% to about 10%, 7.25% to about 10%, 7.5% to about 10%, 7.75% to about 10%, 8% to about 10%, 8.25% to about 10%, 8.5% to about 10%, 8.75% to about 10%, 9% to about 10%, 9.25% to about 10%, 9.5% to about 10%, 9.75% to about 10%, 0.1% to about 1%, 0.1% to about 5%, (w/v).

In an embodiment, the anesthetic agent retains its physical stability in a formulation when the anesthetic agent in formulation: (a) does not precipitate; (b) does not lose activity and/or (c) retains its structure (e.g., as analyzed by HPLC).

The anesthetic agent in a formulation can retain an activity of between about 50% and about 100%, between about 60% and about 100%, between about 70% and about 100, between about 80% and about 100%, or between about 90% and about 100% as compared to a sample of lyophilized anesthetic agent that is reconstituted.

In an embodiment, the anesthetic agent in a formulation has a biological activity of at least 50%, 55%, 60%, 65%, 70%, 75%, 80%, 85%, 90%, 95% or 100% as compared to a lyophilized sample.

In an embodiment, the pH of the pharmaceutical formulation is at least about 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, or 9,

In an embodiment, the pH of the pharmaceutical formulation is from about 3 to about 9, about 4 to about 19, about 5 to about 9, about 6 to about 8, about 6 to about 7, about 6 to about 9, about 5 to about 6, about 5 to about 7, about 5 to about 8, about 4 to about 9, about 4 to about 8, about 4 to about 7, about 4 to about 6, about 4 to about 5, about 3 to about 8, about 3 to about 7, about 3 to about 6, about 3 to about 5, about 3 to about 4, about 7 to about 8, about 7 to about 9, about 7 to about 10.

In an embodiment, the pharmaceutical formulation comprises two or more different anesthetic agents. In another embodiment, the formulation comprises two or more different types of anesthetic agents (e.g., an opioid and a barbiturate).

In an embodiment, the concentration of non-ionic tonicity modifier is about 0.01% to about 10%, 0.02% to about 10%, 0.05% to about 10%, 0.075% to about 10%, 0.2% to about 10%, 0.3% to about 10%, 0.4% to about 10%, 0.5% to about 10%, 0.6% to about 10%, 0.7% to about 10%, 0.8% to about 10%, 0.9% to about 10%, 1% to about 10%, 1.5% to about 10%, 1.75% to about 10%, 2% to about 10%, 2.25% to about 10%, 2.5% to about 10%, 2.75% to about 10%, 3% to about 10%, 3.25% to about 10%, 3.5% to about 10%, 3.75% to about 10%, 4% to about 10%, 4.25% to about 10%, 4.5% to about 10%, 4.75% to about 10%, 5% to about 10%, 5.25% to about 10%, 5.5% to about 10%, 5.75% to about 10%, 6% to about 10%, 6.25% to about 10%, 6.5% to about 10%, 6.75% to about 10%, 7% to about 10%, 7.25% to about 10%, 7.5% to about 10%, 7.75% to about 10%, 8% to about 10%, 8.25% to about 10%, 8.5% to about 10%, 8.75% to about 10%, 9% to about 10%, 9.25% to about 10%, 9.5% to about 10%, 9.75% to about 10%, 0.1% to about 10%, 0.1% to about 5%, 0.1% to about 2%, (w/v).

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Other embodiments of the invention include a stabilizing formulation for an anesthetic, wherein the formulation is administered to a patient topically, orally, rectally, vaginally, parenterally, intrapulmonary, sublingually, pulmonary and or intranasal. Aspects of the present invention disclose a pharmaceutical formulation, wherein the formulation is in the form of a sold or liquid; and further aspects, wherein the formulation is in the form of a liquid, powder, tablet, a capsule, a gel tab, a lozenge, an orally dissolved strip, syrup, an oral suspension, an emulsion, a granule, a sprinkle and a pellet; and further aspects wherein the formulation is a pharmaceutical composition comprising an anesthetic formulated in a pharmaceutical formulation.

In another aspect, a kit is provided comprising an anesthetic formulated in a pharmaceutical formulation; and further aspects, wherein the kit comprises a package containing the pharmaceutical composition and instructions; and further aspects, wherein the kit comprises a package containing the pharmaceutical composition and a device to administer the composition to a human or animal; and further aspects, wherein the device is an injectable device; and further aspects, wherein the injectable device is selected from a syringe, pen injector, auto injector and needle free injectors; and further aspects, wherein the needle free injector is a syringe; and further aspects, wherein the syringe is prefilled with a liquid; and further aspects, wherein the syringe has a single chamber; and further aspects, wherein the syringe has dual chambers; and further aspects, wherein composition is lyophilized.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A stable anesthetic formulation, the formulation comprising:
a) tetracaine,
b) lidocaine,
c) sorbitol at about 5% (w/w),
d) about 10 mM sodium acetate buffer and
e) water.
wherein the formulation has a pH of about 3.5 to 4.5.

2. The formulation of claim 1, wherein the tetracaine is about 0.5% (w/w).

3. The formulation of claim 1, wherein the lidocaine is about 4% (w/w).

4. The formulation of claim 1, further comprising epinephrine.

5. A method of increasing the stability of an anesthetic in solution, the method comprising:
a) providing an anesthetic,
b) adding a non-ionic tonicity modifier, and
c) adjusting the pH of the anesthetic solution to 4.5 or lower.

6. The method of claim 5, wherein the anesthetic is one or more of tetracaine and lidocaine.

7. The method of claim 5, wherein the non-ionic tonicity modifier is comprised of one or more carbohydrates.

8. The method of claim 5, wherein the non-ionic tonicity modifier is sorbitol.

9. The method of claim 5, wherein the pH is about 3.5.

10.-14. (canceled)

15. A stable anesthetic formulation, the formulation comprising:
a) tetracaine,
b) lidocaine,
c) epinephrine,
d) a non-ionic tonicity modifier,
e) about 10 mM sodium acetate buffer and
f) water,
wherein the formulation has a pH of about 3.5 to 4.5.

16. The formulation of claim 15, wherein the non-ionic tonicity modifier is sorbitol.

17. The formulation of claim 15, wherein the concentration of the non-ionic tonicity modifier is about 5 to 10% (w/w).

18. The formulation of claim 15, wherein the concentration of tetracaine is about 5 mg/ml.

19. The formulation of claim 15, wherein the concentration of lidocaine is about 40 mg/ml.

20. (canceled)

21.-28. (canceled)

29. The formulation of claim 15, further comprising a permeation enhancer.

30. (canceled)
